(19) United States
(12) Patent Application Publication
(10) Pub. No.: US 2013/0190368 A1
(43) Pub. Date: Jul. 25, 2013

(54) NOVEL POLYMORPHS OF FEBUXOSTAT

(71) Applicants: Bandi Parthasaradhi Reddy, Hyderabad (IN); Kura Rathnakar Reddy, Hyderabad (IN); Dasari Muralidhara Reddy, Hyderabad (IN); Matta Ramakrishna Reddy, Hyderabad (IN); Bandi Vamsi Krishna, Hyderabad (IN)

(72) Inventors: Bandi Parthasaradhi Reddy, Hyderabad (IN); Kura Rathnakar Reddy, Hyderabad (IN); Dasari Muralidhara Reddy, Hyderabad (IN); Matta Ramakrishna Reddy, Hyderabad (IN); Bandi Vamsi Krishna, Hyderabad (IN)

(73) Assignee: HETERO RESEARCH FOUNDATION, Hyderabad (IN)

(21) Appl. No.: 13/825,689

(22) PCT Filed: Aug. 23, 2011
(86) PCT No.: PCT/IN11/00566

§ 371 Date: Apr. 10, 2013

(30) Foreign Application Priority Data
Sep. 24, 2010 (IN) 2810/CHE/2010

(51) Int. Cl. C07D 277/593

(52) U.S. Cl. CPC C07D 277/593
USPC 514/365; 548/201

(57) ABSTRACT

The present invention provides a novel 1,4-dioxane solvate form of febuxostat and process for its preparation. The present invention also provides novel crystalline forms of febuxostat, processes for their preparation and pharmaceutical compositions comprising them.

### CROSS-REFERENCE TO RELATED APPLICATIONS

The present application a 371 of Indian Patent Application No. 2810/CHE/2010, filed on Sep. 24, 2010 under the provisions of 35 U.S.C. §119 and the International Convention for the protection of Industrial Property, which are incorporated herein by reference in their entirety.

### FIELD OF THE INVENTION

The present invention provides a novel 1,4-dioxane solvate form of febuxostat and process for its preparation. The present invention also provides novel crystalline forms of febuxostat, processes for their preparation and pharmaceutical compositions comprising them.

### BACKGROUND OF THE INVENTION

Febuxostat is chemically, 2-[3-cyano-4-(2-methylpropoxy)phenyl]-4-methyl-5-thiazolecarboxylic acid and has the structural formula:

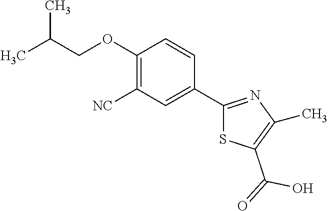 [SMILES: Cc1nc(-c2ccc(OCC(C)C)c(C#N)c2)sc1C(=O)O]

Febuxostat (brand names Adenuric (EU) and Uloric (US)) is an inhibitor of xanthine oxidase that is indicated for use in the treatment of hyperuricemia and gout. The drug is marketed by Menarini. A study comparing febuxostat to allopurinol found that more individuals treated with febuxostat had decreased levels of uric acid, but there was no difference in the amount of initial gout flares or the surface area of gout tophi.

Polymorphism is defined as “the ability of a substance to exist as two or more crystalline phases that have different arrangement and/or conformations of the molecules in the crystal Lattice. Thus, in the strict sense, polymorphs are different crystalline structures of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and Infrared spectrometry (IR).

Solvent medium and mode of crystallization play very important role in obtaining one polymorphic Form over the other.

Febuxostat can exist in different polymorphic forms, which may differ from each other in terms of stability, physical properties, spectral data and methods of preparation.

Febuxostat and its process were disclosed in U.S. Pat. No. 5,614,520.

U.S. Pat. No. 6,225,474 disclosed crystalline form A, form B, form C, form D, form G and amorphous form of febuxostat.

PCT publication no. WO 2008/067773 disclosed crystalline form H, form and form J of febuxostat.

Crystalline form I and form II of febuxostat were disclosed CN patent publication no. 101139325.

CN patent publication no. 101386605 disclosed a crystalline form K of febuxostat, characterized by an X-ray powder diffraction pattern having peaks expressed as 2θ at about 5.64, 7.80, 11.38, 11.70, 12.54, 12.74, 17.18 and 26.12±0.2 degrees.

CN patent publication no. 101412700 disclosed a crystalline form of febuxostat, characterized by an X-ray powder diffraction pattern having peaks expressed as 2θ at about 5.54, 5.66, 7.82, 11.48, 12.62, 16.74, 17.32, 18.04, 18.34, 20.40, 23.74, 25.76 and 26.04±0.2 degrees.

Crystalline form Q of febuxostat was disclosed in CN patent publication no. 101648926.

CN patent publication no. 101671315 disclosed a crystalline form K of febuxostat, characterized by an X-ray powder diffraction pattern having peaks expressed as 2θ at about 4.82, 6.64, 6.88, 7.22, 11.74, 12.82, 13.28, 16.00, 16.50, 17.50, 20.98, 22.02, 23.00, 23.82, 24.70, 25.18, 25.84 and 26.68±0.2 degrees.

Crystalline form X, form Y and form Z of febuxostat were disclosed in CN patent publication no. 101684107.

We have discovered novel 1,4-dioxane solvate form of febuxostat.

The 1,4-dioxane solvate form of the present invention may also serve as intermediate for preparation of febuxostat crystalline form H1, febuxostat crystalline form H2 or other polymorphs of febuxostat.

We have also discovered two novel crystalline forms of febuxostat. The novel forms have been found to be stable over the time and reproducible and so, suitable for pharmaceutical preparations.

Thus, one object of the present invention is to provide a novel 1,4-dioxane solvate form of febuxostat and process for its preparation.

Another object of the present invention is to provide novel crystalline forms of febuxostat, processes for their preparation and pharmaceutical compositions comprising them.

### SUMMARY OF THE INVENTION

In one aspect, the present invention provides a novel 1,4-dioxane solvate form of febuxostat characterized by peaks in the powder x-ray diffraction spectrum having 2θ angle positions at about 4.8, 6.7, 11.5, 15.8 and 25.9±0.2 degrees.

In another aspect, the present invention provides a process for the preparation of febuxostat 1,4-dioxane solvate form, which comprises crystallizing febuxostat from 1,4-dioxane solvent and isolating febuxostat 1,4-dioxane solvate form.

In another aspect, the present invention provides a crystalline form of febuxostat designated as form H1 characterized by peaks in the powder x-ray diffraction spectrum having 2θ angle positions at about 5.7, 7.9, 11.4, 12.6, 17.7, 20.4, 24.6 and 25.7±0.2 degrees.

In another aspect, the present invention provides a process for the preparation of febuxostat crystalline form H1, which comprises:
a) providing a solution of febuxostat in an ester solvent;
b) heating the solution obtained in step (a) at reflux;
c) cooling the reaction mass obtained in step (b) at below 20° C.; and
d) isolating febuxostat crystalline form H1.

In another aspect, the present invention provides a pharmaceutical composition comprising crystalline form H1 of febuxostat and pharmaceutically acceptable excipients.

In another aspect, the present invention provides a crystalline form of febuxostat designated as form H2 characterized by peaks in the powder x-ray diffraction spectrum having 2θ angle positions at about 5.8, 6.5, 11.5, 17.3, 25.8 and 26.6±0.2 degrees.

In another aspect, the present invention provides a process for the preparation of febuxostat crystalline form H2, which comprises:
a) suspending febuxostat in cyclohexane;
b) heating the suspension obtained in step (a) at reflux; and
c) isolating febuxostat crystalline form H2.

In yet another aspect, the present invention provides a pharmaceutical composition comprising crystalline form H2 of febuxostat and pharmaceutically acceptable excipients.

### BRIEF DESCRIPTION OF THE DRAWING

FIG. 1 is an X-ray powder diffraction spectrum of febuxostat 1,4-dioxane solvate form.

FIG. 2 is an X-ray powder diffraction spectrum of febuxostat crystalline form H1.

FIG. 3 is an X-ray powder diffraction spectrum of febuxostat crystalline form H2.

X-ray powder diffraction spectrum was measured on a bruker axs D8 advance X-ray powder diffractometer having a copper-Ka radiation. Approximately 1 gm of sample was gently flattered on a sample holder and scanned from 2 to 50 degrees two-theta, at 0.02 degrees two theta per step and a step time of 10.8 seconds. The sample was simply placed on the sample holder. The sample was rotated at 30 rpm at a voltage 40 KV and current 35 mA.

### DETAILED DESCRIPTION OF THE INVENTION

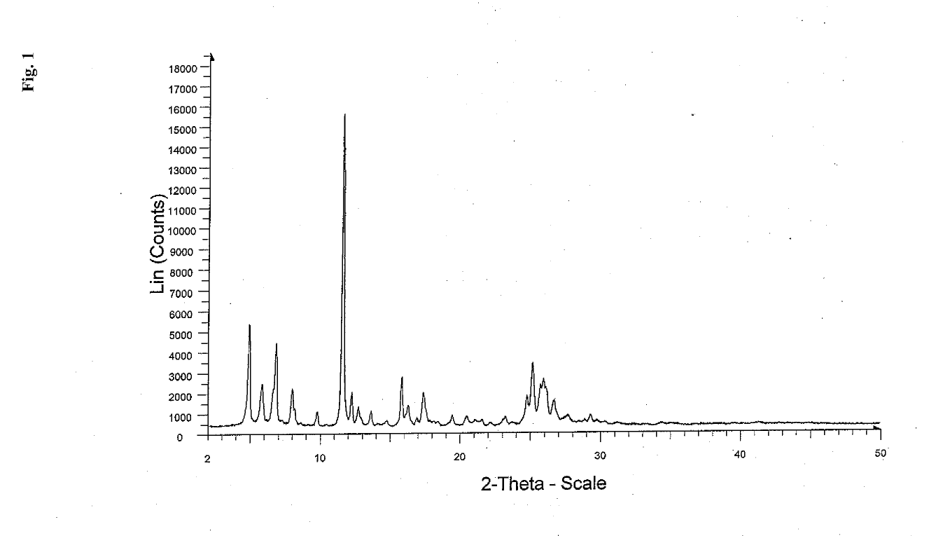

According to one aspect of the present invention, there is provided a novel 1,4-dioxane solvate form of febuxostat characterized by peaks in the powder x-ray diffraction spectrum having 2θ angle positions at about 4.8, 6.7, 11.5, 15.8 and 25.9±0.2 degrees. The powdered x-ray diffractogram (PXRD) of febuxostat 1,4-dioxane solvate form is shown in FIG. 1.

According to another aspect of the present invention, there is provided a process for the preparation of febuxostat 1,4-dioxane solvate form, which comprises crystallizing febuxostat from 1,4-dioxane solvent and isolating febuxostat 1,4-dioxane solvate form.

Febuxostat used in the process may preferably be any other polymorphic forms. Thus, for example, febuxostat crystalline form G, febuxostat crystalline form A or febuxostat crystalline form C.

Febuxostat 1,4-dioxane solvate form may be isolated in the process by methods known such as filtration or centrifugation.

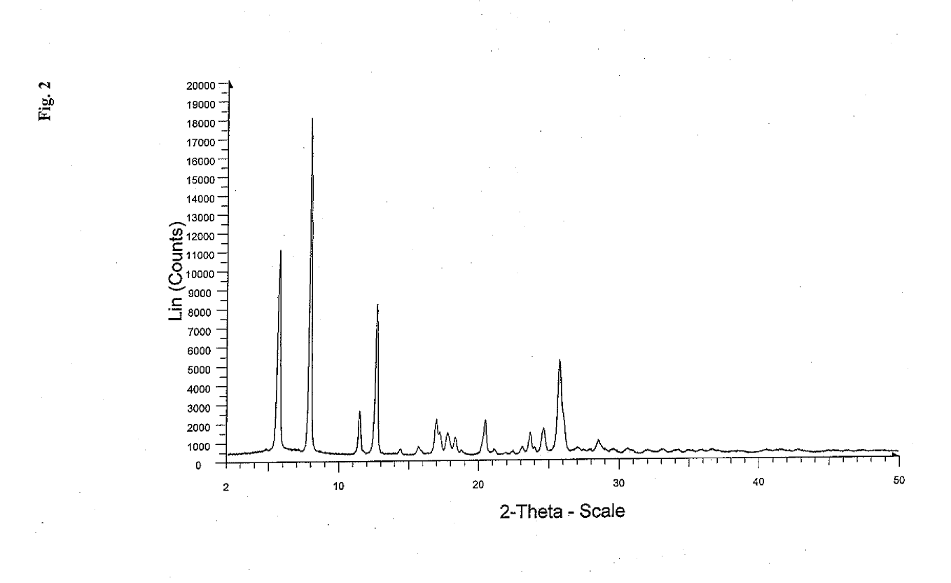

According to another aspect of the present invention, there is provided a crystalline form of febuxostat designated as form H1 characterized by peaks in the powder x-ray diffraction spectrum having 2θ angle positions at about 5.7, 7.9, 11.4, 12.6, 17.7, 20.4, 24.6 and 25.7±0.2 degrees. The powdered x-ray diffractogram (PXRD) of febuxostat crystalline form H1 is shown in FIG. 2.

The febuxostat crystalline form H1 may be identified and differentiated from the known polymorphs by its characteristic PXRD pattern. Thus, for example, a peak at 5.54 degrees 2θ is absent in the PXRD of the febuxostat crystalline form H1 of the present invention, but is present in the PXRD of the crystalline form of febuxostat disclosed in the CN patent publication no. 101412700.

According to another aspect of the present invention, there is provided a process for the preparation of febuxostat crystalline form H1, which comprises:
a) providing a solution of febuxostat in an ester solvent;
b) heating the solution obtained in step (a) at reflux;
c) cooling the reaction mass obtained in step (b) at below 20° C.; and
d) isolating febuxostat crystalline form H1.

Febuxostat used in step (a) may preferably be any other polymorphic forms. Thus, for example, febuxostat 1,4-dioxane solvate form of the invention, febuxostat crystalline form G, febuxostat crystalline form H2 of the invention, febuxostat crystalline form A or febuxostat crystalline form C.

The ester solvent used in step (a) may preferably be a solvent or mixture of solvents selected from ethyl acetate, methyl acetate, isopropyl acetate, tert-butyl methyl acetate and ethyl formate, and more preferably the ester solvent is ethyl acetate.

The step (c) may preferably be carried out at about 0 to 10° C., and more preferably at about 0 to 5° C.

Febuxostat crystalline form H1 may be isolated in step (d) by methods known such as filtration or centrifugation.

According to another aspect of the present invention, there is provided a pharmaceutical composition comprising crystalline form H1 of febuxostat and pharmaceutically acceptable excipients, and optionally other therapeutic ingredients.

The crystalline form H1 may preferable be formulated into tablets, capsules, suspensions, dispersions, injectables and other pharmaceutical forms.

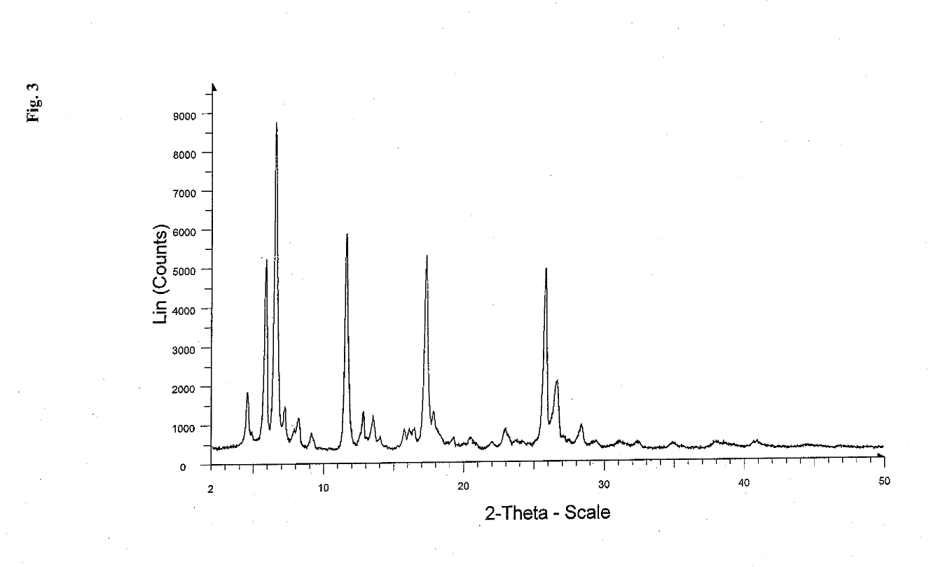

According to another aspect of the present invention, there is provided a crystalline form of febuxostat designated as form H2 characterized by peaks in the powder x-ray diffraction spectrum having 2θ angle positions at about 5.8, 6.5, 11.5, 17.3, 25.8 and 26.6±0.2 degrees. The powdered x-ray diffractogram (PXRD) of febuxostat crystalline form H2 is shown in FIG. 3.

According to another aspect of the present invention, there is provided a process for the preparation of febuxostat crystalline form H2, which comprises:
a) suspending febuxostat in cyclohexane;
b) heating the suspension obtained in step (a) at reflux; and
c) isolating febuxostat crystalline form H2.

Febuxostat used in step (a) may preferably be any other polymorphic forms. Thus, for example, febuxostat 1,4-dioxane solvate form of the invention, febuxostat crystalline form G, febuxostat crystalline form H1 of the invention, febuxostat crystalline form A or febuxostat crystalline form C.

Isolation of febuxostat crystalline form H2 in step (c) can be performed by conventional methods such as cooling, removal of solvents, concentrating the reaction mass, adding an anti-solvent, extraction with a solvent and the like.

According to another aspect of the present invention, there is provided a pharmaceutical composition comprising crystalline form H2 of febuxostat and pharmaceutically acceptable excipients, and optionally other therapeutic ingredients. The crystalline form H2 may preferable be formulated into tablets, capsules, suspensions, dispersions, injectables and other pharmaceutical forms.

The invention will now be further described by the following examples, which are illustrative rather than limiting.

### EXAMPLES

### Example 1

### Preparation of Febuxostat

2-(3-Cyano-4-isobutoxyphenyl)-4-methylthiazole-5-carboxylic acid ethyl ester (100 gm) was dissolved in ethanol (500 ml) at room temperature and then added a solution of sodium hydroxide (15 gm) in water (30 ml). The temperature of the reaction mass was raised to 60° C. and maintained for 1 hour at 60° C., and then concentrated to obtain a residual mass. To the residual mass was added water (500 ml) and then added ethyl acetate (500 ml). The pH of the reaction mass was adjusted to 2.0 with hydrochloric acid (15%) and then the layers were separated. The aqueous layer was extracted with ethyl acetate and the combined organic layer was treated with carbon. The ethyl acetate solvent was distilled off under vacuum at below 50° C. to obtain a residual mass. To the residual mass was added ethyl acetate (500 ml) and then heated to reflux to obtain a solution. The solution was then cooled to room temperature and stirred for 2 hour at room temperature. The contents were further cooled to 10 to 15° C. and stirred for 2 hour, filtered. The solid obtained was dried to give 84 gm of febuxostat.

### Example 2

### Preparation of Febuxostat 1,4-dioxane Solvate Form

Febuxostat (15 gm) as obtained in example 1 was dissolved in 1,4-dioxane (75 ml) and then heated to 60 to 65° C. to obtain a solution. The solution was then cooled to 0 to 5° C. and stirred for 1 hour at 0 to 5° C. The solid obtained was collected by filtration and dried under vacuum at below 80° C. for 8 hours to obtain 8 gm of febuxostat 1,4-dioxane solvate form.

### Example 3

### Preparation of Febuxostat Crystalline Form H1

Febuxostat (15 gm) was dissolved in ethyl acetate (225 ml) and then heated to reflux to obtain a solution. The solution was then cooled to 0 to 5° C. and stirred for 1 hour at 0 to 5° C., filtered. The solid obtained was dried under vacuum at below 80° C. for 8 hours to obtain 13 gm of febuxostat crystalline form H1.

### Example 4

### Preparation of Febuxostat Crystalline form H1

Febuxostat 1,4-dioxane solvate form (500 gm) as obtained in example 2 was dissolved in ethyl acetate (7400 ml) and then heated to reflux to obtain a solution. The solution was then cooled to 0 to 5° C. and stirred for 1 hour at 0 to 5° C. The solid obtained was collected by filtration and dried under vacuum at below 80° C. for 9 hours to obtain 250 gm of febuxostat crystalline form H1.

### Example 5

### Preparation of Febuxostat Crystalline Form H2

Febuxostat (15 gm) was suspended in cyclohexane (300 ml) at room temperature. The contents were heated to reflux and maintained for 1 hour at reflux to obtain a solution. The solution was then cooled to room temperature and stirred for 1 hour at room temperature. The solid obtained was collected by filtration and dried under vacuum at below 80° C. for 8 hours to obtain 12 gm of febuxostat crystalline form H2.

### Example 6

### Preparation of Febuxostat Crystalline Form H2

Febuxostat 1,4-dioxane solvate form (5 gm) was suspended in cyclohexane (100 ml) at room temperature. The contents were heated to reflux and maintained for 1 hour at reflux to obtain a solution. The solution was then cooled to room temperature and stirred for 1 hour at room temperature, filtered. The solid obtained was dried to obtain 3 gm of febuxostat crystalline form H2.

### Example 7

### Preparation of Febuxostat Crystalline Form H1

Example 3 was repeated using isopropyl acetate solvent instead of ethyl acetate solvent to obtain febuxostat crystalline form H1.

### Example 8

### Preparation of Febuxostat Crystalline Form H1

Example 3 was repeated using tert-butyl methyl acetate solvent instead of ethyl acetate solvent to obtain febuxostat crystalline form H1.

### Example 9

### Preparation of Febuxostat Crystalline Form H1

Example 4 was repeated using febuxostat crystalline form H2 as obtained in example 5 instead of febuxostat 1,4-dioxane solvate form to obtain febuxostat crystalline form H1.

### Example 10

### Preparation of Febuxostat Crystalline Form H2

Example 6 was repeated using febuxostat crystalline form G instead of febuxostat 1,4-dioxane solvate form to obtain febuxostat crystalline form H2.

### Example 11

### Preparation of Febuxostat Crystalline Form H2

Example 6 was repeated using febuxostat crystalline form C instead of febuxostat 1,4-dioxane solvate form to obtain febuxostat crystalline form H2.

### Example 12

### Preparation of Febuxostat Crystalline Form H2

Example 6 was repeated using febuxostat crystalline form H1 as obtained in example 3 instead of febuxostat 1,4-dioxane solvate form to obtain febuxostat crystalline form H2.

1. A febuxostat 1,4-dioxane solvate form, characterized by peaks in the powder x-ray diffraction spectrum having 2θ angle positions at about 4.8, 6.7, 11.5, 15.8 and 25.9±0.2 degrees.

2. A febuxostat 1,4-dioxane solvate form, characterized by an x-ray powder diffractogram as shown in FIG. 1.

3. A process for the preparation of febuxostat 1,4-dioxane solvate form as claimed in claim 1, which comprises crystallizing febuxostat from 1,4-dioxane solvent and isolating febuxostat 1,4-dioxane solvate form.

4. A febuxostat crystalline form H1, characterized by peaks in the powder x-ray diffraction spectrum having 2θ angle positions at about 5,7, 7.9, 11,4, 12.6, 17.7, 20.4, 24.6 and 25.7±0.2 degrees.

5. A febuxostat crystalline form H1, characterized by an x-ray powder diffractogram as shown in FIG. 2.

6. A process for the preparation of febuxostat crystalline form H1 as claimed in claim 4, which comprises:
a. providing a solution of febuxostat in an ester solvent;
b. heating the solution obtained in step (a) at reflux;
c. cooling the reaction mass obtained in step (b) at below 20° C.; and
d. isolating febuxostat crystalline form H1.

7. The process as claimed in claim 6, wherein the ester solvent used in step (a) is a solvent or mixture of solvents selected from ethyl acetate, methyl acetate, isopropyl acetate, tert-butyl methyl acetate and ethyl formate.

8. The process as claimed in claim 7, wherein the ester solvent is ethyl acetate.

9. The process as claimed in claim 6, wherein the step (c) is carried out at about 0 to 10° C.

10. The process as claimed in claim 9, wherein the step (c) is carried out at about 0 to 5° C.

11. A febuxostat crystalline form H2, characterized by peaks in the powder x-ray diffraction spectrum having 2θ angle positions at about 5.8, 6.5, 11.5, 17.3, 25.8 and 26.6±0.2 degrees.

12. A febuxostat crystalline form H2, characterized by an x-ray powder diffractogram as shown in FIG. 3.

13. A process for the preparation of febuxostat crystalline form H2 as claimed in claim 11, which comprises:
a. suspending febuxostat in cyclohexane;
b. heating the suspension obtained in step (a) at reflux; and
c. isolating febuxostat crystalline form H2.

14. A pharmaceutical composition that comprises crystalline form H1 of febuxostat and pharmaceutically acceptable excipients, and optionally other therapeutic ingredients.

15. A pharmaceutical composition that comprises crystalline form H2 of febuxostat and pharmaceutically acceptable excipients, and optionally other therapeutic ingredients.

16. The pharmaceutical composition as claimed in claim 14, wherein the polymorphic forms are formulated into tablets, capsules, suspensions, dispersions, injectables and other pharmaceutical forms.

17. The pharmaceutical composition as claimed in claim 15, wherein the polymorphic forms are formulated into tablets, capsules, suspensions, dispersions, injectables and other pharmaceutical forms.
